SEQUENCE LISTING (57 sequences)

SEQ ID NO: 1 - DNA, 20 bp, Artificial Sequence
  gRNA 1
gtcctgatatttaatgaggc

SEQ ID NO: 2 - DNA, 20 bp, Artificial Sequence
  gRNA 2
gtcctaagaccaccctgagc

SEQ ID NO: 3 - DNA, 20 bp, Artificial Sequence
  gRNA 3
gagttgatggtttttagaac

SEQ ID NO: 4 - DNA, 20 bp, Artificial Sequence
  gRNA 4
gtaagagatacatctaacat

SEQ ID NO: 5 - DNA, 20 bp, Artificial Sequence
  gRNA 5
gtttgtacttataactaggc

SEQ ID NO: 6 - DNA, 20 bp, Artificial Sequence
  gRNA 6
gtgtatgaatatagatacat

SEQ ID NO: 7 - DNA, 24 bp, Artificial Sequence
  g1 Forward oligonucleotide
caccgtcctgatatttaatgaggc

SEQ ID NO: 8 - DNA, 24 bp, Artificial Sequence
  g1 Reverse oligonucleotide
aaacgcctcattaaatatcaggac

SEQ ID NO: 9 - DNA, 24 bp, Artificial Sequence
  g2 Forward oligonucleotide
caccgtcctaagaccaccctgagc

SEQ ID NO: 10 - DNA, 24 bp, Artificial Sequence
  g2 Reverse oligonucleotide
aaacgctcagggtggtcttaggac

SEQ ID NO: 11 - DNA, 24 bp, Artificial Sequence
  g3 Forward oligonucleotide
caccgagttgatggtttttagaac

SEQ ID NO: 12 - DNA, 24 bp, Artificial Sequence
  g3 Reverse oligonucleotide
aaacgttctaaaaaccatcaactc

SEQ ID NO: 13 - DNA, 24 bp, Artificial Sequence
  g4 Forward oligonucleotide
caccgtaagagatacatctaacat

SEQ ID NO: 14 - DNA, 24 bp, Artificial Sequence
  g4 Reverse oligonucleotide
aaacatgttagatgtatctcttac

SEQ ID NO: 15 - DNA, 24 bp, Artificial Sequence
  g5 Forward oligonucleotide
caccgtttgtacttataactaggc

SEQ ID NO: 16 - DNA, 24 bp, Artificial Sequence
  g5 Reverse oligonucleotide
aaacgcctagttataagtacaaac

SEQ ID NO: 17 - DNA, 24 bp, Artificial Sequence
  g6 Forward oligonucleotide
caccgtgtatgaatatagatacat

SEQ ID NO: 18 - DNA, 24 bp, Artificial Sequence
  g6 Reverse oligonucleotide
aaacatgtatctatattcatacac

SEQ ID NO: 19 - DNA, 18 bp, Artificial Sequence
  U6 promoter Forward oligonucleotide
gagggcctatttcccatg

SEQ ID NO: 20 - DNA, 24 bp, Artificial Sequence
  T7E1 assay First Forward primer
cttctgtcctcatcattggtaacg

SEQ ID NO: 21 - DNA, 21 bp, Artificial Sequence
  T7E1 assay First Reverse primer
acatctgagaaggcacctcac

SEQ ID NO: 22 - DNA, 20 bp, Artificial Sequence
  T7E1 assay Second Forward primer
ggcattggcttctttagggc

SEQ ID NO: 23 - DNA, 24 bp, Artificial Sequence
  T7E1 assay Second Reverse primer
gtgtcacttaaaatagtaggtccc

SEQ ID NO: 24 - DNA, 22 bp, Artificial Sequence
  5′ Junction PCR Outer-FP primer
tccaataaaataagcgggtgac

SEQ ID NO: 25 - DNA, 20 bp, Artificial Sequence
  5′ Junction PCR Donor-RP2 primer
ggcgccctggtttacataag

SEQ ID NO: 26 - DNA, 20 bp, Artificial Sequence
  3′ Junction PCR Donor-FP6 primer
gttcccatctcaaggcacac

SEQ ID NO: 27 - DNA, 20 bp, Artificial Sequence
  3′ Junction PCR Outer-RP primer
tccaccattctcccactgac

SEQ ID NO: 28 - DNA, 23 bp, Artificial Sequence
  3′ A (FC 13) Donor-FP primer
tcaaggtcaaaatcgtcaagagc

SEQ ID NO: 29 - DNA, 20 bp, Artificial Sequence
  3′ A (FC 13) Outer-RP primer
tccaccattctcccactgac

SEQ ID NO: 30 - DNA, 21 bp, Artificial Sequence
  3′ B (FC 13) Donor-FP3 primer
tcctctcgttgtgcatagtcg

SEQ ID NO: 31 - DNA, 20 bp, Artificial Sequence
  3′ B (FC 13) Outer-RP primer
tccaccattctcccactgac

SEQ ID NO: 32 - DNA, 20 bp, Artificial Sequence
  3′ C (FC 13) Donor-FP5 primer
ccagctcgcagacctacatg

SEQ ID NO: 33 - DNA, 20 bp, Artificial Sequence
  3′ C (FC 13) Outer-RP primer
tccaccattctcccactgac

SEQ ID NO: 34 - DNA, 23 bp, Artificial Sequence
  Donor D (FC 13) Donor-FP primer
tcaaggtcaaaatcgtcaagagc

SEQ ID NO: 35 - DNA, 20 bp, Artificial Sequence
  Donor D (FC 13) Donor-RP4 primer
catgtgtgagaggggcagtg

SEQ ID NO: 36 - DNA, 22 bp, Artificial Sequence
  5′ E (FUC 11) Outer-FP primer
tccaataaaataagcgggtgac

SEQ ID NO: 37 - DNA, 20 bp, Artificial Sequence
  5′ E (FUC 11) Donor-RP3 primer
gtttgaatgcatgggagagc

SEQ ID NO: 38 - DNA, 20 bp, Artificial Sequence
  OCT4 Forward primer
ttgggctcgagaaggatgtg

SEQ ID NO: 39 - DNA, 21 bp, Artificial Sequence
  OCT4 Reverse primer
tcctctcgttgtgcatagtcg

SEQ ID NO: 40 - DNA, 20 bp, Artificial Sequence
  SOX2 Forward primer
gccctgcagtacaactccat

SEQ ID NO: 41 - DNA, 19 bp, Artificial Sequence
  SOX2 Reverse primer
tgccctgctgcgagtagga

SEQ ID NO: 42 - DNA, 20 bp, Artificial Sequence
  KLF4 Forward primer
cgccgctccattaccaagag

SEQ ID NO: 43 - DNA, 20 bp, Artificial Sequence
  KLF4 Reverse primer
cacgatcgtcttcccctctt

SEQ ID NO: 44 - DNA, 20 bp, Artificial Sequence
  GAPDH Forward primer
gtggacctgacctgccgtct

SEQ ID NO: 45 - DNA, 20 bp, Artificial Sequence
  GAPDH Reverse primer
ggaggagtgggtgtcgctgt

SEQ ID NO: 46 - DNA, 23 bp, Artificial Sequence
  Endo-OCT4 Forward primer
gacagggggaggggaggagctag

SEQ ID NO: 47 - DNA, 25 bp, Artificial Sequence
  Endo-OCT4 Reverse primer
cttccctccaaccagttgccccaaa

SEQ ID NO: 48 - DNA, 20 bp, Artificial Sequence
  Endo-SOX2 Forward primer
tggcgaaccatctctgtggt

SEQ ID NO: 49 - DNA, 20 bp, Artificial Sequence
  Endo-SOX2 Reverse primer
ccaacggtgtcaacctgcat

SEQ ID NO: 50 - DNA, 26 bp, Artificial Sequence
  Endo-KLF4 Forward primer
acagtctgttatgcactgtggtttca

SEQ ID NO: 51 - DNA, 27 bp, Artificial Sequence
  Endo-KLF4 Reverse primer
catttgttctgcttaaggcatacttgg

SEQ ID NO: 52 - DNA, 19 bp, Artificial Sequence
  c-MYC
ccagcagcgactctgagga

SEQ ID NO: 53 - DNA, 21 bp, Artificial Sequence
  c-MYC Reverse primer
gagcctgcctcttttccacag

SEQ ID NO: 54 - DNA, 25 bp, Artificial Sequence
  NANOG
caagaaacagaagaccagaactgtg

SEQ ID NO: 55 - DNA, 24 bp, Artificial Sequence
  NANOG Reverse primer
ccattgctattcttcggccagttg

SEQ ID NO: 56 - DNA, 20 bp, Artificial Sequence
  GAPDH
gtggacctgacctgccgtct

SEQ ID NO: 57 - DNA, 20 bp, Artificial Sequence
  GAPDH Reverse primer
ggaggagtgggtgtcgctgt

(19) United States
(12) Patent Application Publication
(10) Pub. No.: US 2024/0093243 A1
(43) Pub. Date: Mar. 21, 2024

(54) METHOD FOR PRODUCING INDUCED PLURIPOTENT STEM CELL BY USING CRISPR/CAS SYSTEM

(71) Applicant: AJOU UNIVERSITY INDUSTRY-ACADEMIC COOPERATION FOUNDATION, Gyeonggi-do (KR)

(72) Inventors: Sangdun CHOI, Suwon-si (KR); Javaid NASIR, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 18/268,894

(22) PCT Filed: Dec. 23, 2021
(86) PCT No.: PCT/KR2021/019696

§ 371 Date: Jun. 21, 2023

(30) Foreign Application Priority Data
Dec. 23, 2020 (KR) 10-2020-0181723

(51) Int. Cl. C12N 15/90; C07K 14/47; C12N 9/22; C12N 15/11

(52) U.S. Cl. CPC C12N 15/907; C07K 14/47; C12N 9/22; C12N 15/111; C12N 2310/20; C12N 2800/80

(57) ABSTRACT

A method for producing induced pluripotent stem cells by using CRISPR/Cas system is disclosed. The method includes a step of inserting a reprogramming factor by using a CRSPR/Cas system containing in a safe harbor of somatic cells a delivery means including two guides RNAs and a Cas protein-encoding nucleic acid. The method can prevent mutation in an off-target region and integrate a reprogramming factor into an on-target region safely and specifically, and regulates the reprogramming factor with doxycycline to guarantee consistent expression, whereby induced pluripotent stem cells can be effectively produced. In addition, the induced pluripotent stem cells produced by the method exhibit multipotent markers similar to those of human embryonic stem cells and can differentiate into derivatives of all the three germ layers.

### TECHNICAL FIELD

The present invention relates to a method of producing induced pluripotent stem cells using a CRISPR/Cas system, and more particularly to a method of producing induced pluripotent stem cells including inserting a reprogramming factor into a safe harbor of somatic cells using a CRISPR/Cas system containing a delivery vehicle including nucleic acids encoding two guide RNAs and a Cas protein.

### BACKGROUND ART

Induced pluripotent stem cells (iPSCs) are produced by ectopic expression of reprogramming factors through genome-integrative and genome-nonintegrative approaches. Non-integrative methods (no integration of foreign DNA into the genome) are considered safer, but they are slow and inefficient. Although integrative approaches are considered effective in ensuring consistent expression of reprogramming factors, reported viral and non-viral integrative approaches suffer from random and uncontrolled copy number of integrated sequences, insertional mutations, and/or reactivation of reprogramming factors (Gonzalez, F., et al., Nature Reviews Genetics 12, 231-242 (2011); Malik, N. & Rao, M. S. in Pluripotent stem cells 23-33 (Springer, 2013)). Therefore, there is a need for research into methods of producing iPSCs for personalized medical treatment in disease modeling, drug screening, and cell-based therapy (Tabar, V. & Studer, L. Nature Reviews Genetics 15, 82 (2014); Ebert, A. D. & Svendsen, C. N. Nature Reviews Drug Discovery 9, 367 (2010)).

Meanwhile, in order to avoid random integration of DNA encoding reprogramming factors, the host genome must have a genomic safe harbor (GSH). To date, AAVS1, CCR5, and hROSA26 have been widely used as GSHs, but they have the following limitations. For example, integration of a cryptic splice acceptor containing transgene into the AAVS1 locus (locus 19q13.42) may cause expression of the adjacent gene (phosphatase 1 regulatory subunit 12C; PPP1R12C) to be reduced by 50% to 100%. Genetic studies in humans have revealed that CCR5 (locus 3p21.31) homozygous knockout may increase the risk of infection by West Nile Virus. Moreover, the CCR5 locus results in lower expression of integrated transgenes compared to the AAVS1 locus. Like other GSHs, hROSA26 (locus 3p25.3) may also express transgenes, but all three GSHs are located in gene-rich regions for which safety data are lacking. Furthermore, they do not satisfy the complete criteria for GSH selection: (1) a >50 kbp distance from the 5′ end of a gene, (2) a >300 kbp distance from any cancer-associated gene, (3) a >300 kbp distance from any microRNA gene, and (4) locations outside a superconserved sequence and a gene transcription unit (Papapetrou, E. P. et al. Nature Biotechnology 29, 73 (2011)).

Papapetrou, et al. used a lentiviral system and reported three regions of the human genome (chromosome 1 (position 188,083,272), chromosome 10 (position 3,046,320), and chromosome 17 (position 67,328,980)) satisfying GSH criteria (Papapetrou, E. P. et al. Nature Biotechnology 29, 73 (2011)). Despite identification through lentiviral systems, these regions are problematic in that they are not specifically targeted by traditional genome editing tools due to the following limitations.

Currently, various site-specific nucleases such as zinc-finger nucleases (ZFNs), transcription activator-like effector nucleases (TALENs), and CRISPR/Cas9 are being used as genome-engineering tools, but ZFNs and TALENs create double-strand breaks (DSBs) by an artificial nuclease domain (FokI) and require repeated work. On the other hand, the CRISPR/Cas9 system is the best and most efficient system that performs genome editing via bacterial type II with clustered regularly interspaced short palindromic repeat (CRISPR) and CRISPR-associated protein 9 (Cas 9). In order to generate a double-strand break (DSB), this system requires a 20-nucleotide-long CRISPR RNA that must be complementary to a target sequence upstream of a 5′ protospacer adjacent motif (PAM) site. The resulting DSB is repaired by non-homologous end joining (NHEJ) or homology-directed repair (HDR); and the latter may be used to insert any gene of interest into the target site. Also, a CRISPR/Cas9 system using single gRNA may tolerate specific mismatches to DNA targets, promoting unwanted off-target mutagenesis, whereas a double-nicking CRISPR/Cas9 system using paired gRNAs is suitable for genome editing with increased specificity and minimized off-target mutagenesis by expanding the number of bases specifically recognized at the target site (F. Ann Ran et al., Double nicking by RNA-guided CRISPR Cas9 for enhanced genome editing specificity, Cell. 2013 September 12; 154(6): 1380-1389).

However, since the efficiency of the homology-directed-repair pathway for knock-in of large transgene(s) at the target site is low, a method of producing iPSCs by integrating reprogramming factors into CASH-1 (CRISPR/Cas9-accessible safe harbor-1) using the CRISPR/Cas9 system has not yet been reported (Yang, H. et al. Cell 154, 1370-1379 (2013); Mali, P. et al. Science 339, 823-826 (2013)).

Against this technical background, the present inventors have made great efforts to integrate reprogramming factors into somatic cells very precisely and safely, and ascertained that induced pluripotent stem cells may be efficiently produced by transfecting CASH-1 (position 188,083,272 of chromosome 1) of somatic cells with a vector including a nucleic acid encoding a reprogramming factor using a double-nicking CRISPR/Cas9 system containing a vector including gRNA (guide RNA) 1 and gRNA 2, thus culminating in the present invention.

The above information described in the background section is only for improving the understanding of the background of the present invention, and it does not include information forming the prior art known to those of ordinary skill in the art to which the present invention pertains.

### Disclosure

It is an object of the present invention to provide a method of producing induced pluripotent stem cells using a CRISPR/Cas system containing a delivery vehicle including nucleic acids encoding two guide RNAs and a Cas protein in order to safely and efficiently insert a reprogramming factor into somatic cells.

In order to accomplish the above object, the present invention provides a method of producing induced pluripotent stem cells including inserting a nucleic acid encoding a reprogramming factor into CASH-1 (CRISPR/Cas9-accessible safe harbor-1) of somatic cells using a CRISPR/Cas system, in which the CRISPR/Cas system contains a delivery vehicle including nucleic acids encoding a guide RNA (gRNA) and a Cas protein, and the gRNA includes gRNA 1 represented by SEQ ID NO: 1 and gRNA 2 represented by SEQ ID NO: 2.

### DESCRIPTION OF DRAWINGS

FIG. 1 shows design, cloning, and experimental validation of CASH-1-specific gRNAs, FIG. 1a showing colony PCR confirmation of gRNAs cloned into pX330-U6-Chimeric_BB-CBh-hSpCas9, FIG. 1b showing confirmation by Sanger sequencing of gRNAs cloned into pX330-U6-Chimeric_BB-CBh-hSpCas9, FIG. 1c showing a T7E1 endonuclease assay after transfection of relevant gRNAs into HEK293T cells, in which indel percentages were calculated using ImageJ software, FIG. 1d showing colony PCR confirmation of gRNAs cloned into pX335-U6-Chimeric_BB-CBh-hSpCas9n(D10A), and FIG. 1e showing confirmation by Sanger sequencing of gRNAs cloned into pX335-U6-Chimeric_BB-CBh-hSpCas9n(D10A), in which each number in colony PCR (a, d) represents the relevant gRNA, the uppercase letter represents a colony name, and the lowercase “n” represents pX335-U6-Chimeric_BB-CBh-hSpCas9n(D10A), L: 100 bp ladder;

FIG. 2 shows CRISPR/Cas9-mediated knock-in of a reprogramming donor cassette characterized by inducible expression, FIG. 2a showing design of a DOX-inducible polycistronic expression cassette of reprogramming factors OCT4, SOX2, and KLF4, in which individual components are named and labeled with different colors, the red arrow below the cassette indicates a restriction site, the cassette is flanked by the sequence homologous to CASH-1, and the DNA sequence of the entire cassette is shown in FIG. 6, FIG. 2b showing detection of GFP signals in donor-transfected HEK293T cells with or without DOX induction, scale bar: 500 μm, FIG. 2c showing confirmation of protein expression of reprogramming factors in donor-transfected HEK293T cells with or without DOX induction, FIG. 2d showing optimization of donor cassette knock-in in HEK293T cells in the table, FIGS. 2e and 2f showing 5′ (e) and 3′ (f) junction PCR assays of template genomic DNA isolated from the selected HEK293T colonies, FIG. 2g showing optimization of donor cassette knock-in in HDFs in the table, FIG. 2h showing 5′ (e) and 3′ (f) junction PCR assays of template genomic DNA isolated from the selected HDF colonies, FIGS. 2i and 2j showing confirmation of mRNA expression of each reprogramming factor in HEK293T-OSK cells (i) and HDF-OSK cells (j) compared to wild-type cells (WT) where GAPDH mRNA served as a loading control, and FIG. 2k showing confirmation of GFP expression in HDF-OSK cells with or without DOX induction for 24 hours, scale bar: 100 μm, L: 1 kbp ladder;

FIG. 3 shows construction and characterization of iPSCs, FIG. 3a showing the workflow for production of iPSCs from HDF-OSK and HEK293T-OST cells using reprogramming medium, FIG. 3b showing the morphological changes during expansion in maintenance medium after reprogramming HDF-OSK and HEK293T-OSK cells into iPSCs, scale bar: 100 μm, FIG. 3c showing the expression levels of pluripotent markers in the produced iPSC clones compared to human embryonic stem cells (hESCs), FIGS. 3d and 3e showing immunofluorescence analysis of pluripotent markers in iPSC clones derived from HDF-OSK cells (d) or HEK 293T-OSK cells (e), scale bar: 100 μm, and FIG. 3f showing silencing of transgenes in morphologically changed HDF-OSK colonies, scale bar: 200 μm;

FIG. 4 shows the differentiation potential of iPSCs, FIG. 4a showing the formation of embryoid bodies through a hanging drop process, scale bar: 100 μm, FIGS. 4b and 4c show immunofluorescence staining of spontaneously differentiating embryoid bodies of HDF-OSK (b) or HEK293T-OSK (c) clones for markers of respective germ layers, particularly a-fetoprotein (AFP) for endoderm, α-smooth muscle actin (α-SMA) for mesoderm, and β III tubulin (TUJ-1) for ectoderm, scale bar: 25 μm, FIG. 4d showing the formation of tumor masses after injection of iPSC clones derived from HEK293T-OSK cells or HDF-OSK cells, in which a negative control is based on parental HEK 293T cells, and FIG. 4e showing immunohistochemical analysis of tissue sections of teratomas formed using antibodies specific to individual germ layers, in which nuclei were stained with Hoechst 33258 solution and images were captured using a fluorescence microscope (Olympus IX53; Olympus Corporation, Tokyo, Japan), scale bar: 250 μm;

FIG. 5 shows the CASH-1 sequence and target sites, in which the marked sequence of chromosome 1 constitutes positions 188,082,217-188,083,803, where the CASH-1 site (188,083,272) is underlined, target sites throughout CASH-1 for designing gRNA are represented in bold with the PAM sequence (5′-NGG-3′) highlighted in red, and target sites for the first and second primer sets required for T7 endonuclease I assay are highlighted in yellow and green, respectively;

FIG. 6 shows the DNA sequence of the reprogramming-donor cassette, in which the design (top) and DNA sequence (bottom) of the polycistronic DOX-inducible expression cassette are illustrated, individual components are named and labeled with different colors, the red arrow below the cassette indicates a restriction site, and the cassette is flanked by the sequence homologous to CASH-1, and abbreviations are as follows: LHS, left-hand homologous sequence; SV40 poly A, simian virus 40 polyadenylation signal; mPGK promoter, mouse phosphoglycerate kinase promoter; TetO, tetracycline operator; OCT4, octamer-binding transcription factor 4; SOX2, sex-determining region Y-box 2; KLF4, Kruppel-like factor 4; EGFP, enhanced green fluorescent protein; bGH poly A, bovine growth hormone (bGH) poly A signal; BSD, blasticidin; RHS, right-hand homologous sequence;

FIG. 7 shows HDF-OSK clone confirmation by junction PCR, FIG. 7a showing 3′ junction PCR to identify HDF-OSK clones using three sets of primers in reactions A, B, and C (FIG. 7c), and FIG. 7b showing donor-specific PCR (Rx D) and 5′ junction PCR (Rx E) to identify HDF-OSK clones using specific primer sets (FIG. 7c), L: 1 kbp ladder, Rx: reaction;

FIG. 8 shows enrichment of GFP-positive cells, FIGS. 8a and 8b showing sorting of DOX-induced HEK293T-OSK cells (a) and HDF-OSK cells (b) based on GFP signal intensity during flow cytometry (FACSAria III, BD Biosciences), in which data processing was performed using FACSDiva software (BD Biosciences);

FIG. 9 shows full blots of RT-PCR for reprogramming factors, FIGS. 9a and 9b showing confirmation of mRNA expression of each reprogramming factor in HEK293T-OSK cells (a) and HDF-OSK cells (b) compared to wild-type cells (WT) where GAPDH mRNA served as a loading control, L: 1 kbp ladder; and

FIG. 10 shows immunofluorescence staining of the negative control, FIGS. 10a and 10b showing immunofluorescence analysis of pluripotent markers in HDF (a) and HEK293T (b), scale bar: 100 μm.

### MODE FOR INVENTION

Unless otherwise defined, all technical and scientific terms used herein have the same meanings as typically understood by those skilled in the art to which the present invention belongs. In general, the nomenclature used herein is well known in the art and is typical.

In an embodiment of the present invention, induced pluripotent stem cells were produced by transfecting CASH-1 (chromosome 1: 188,083,272) of somatic cells with a vector including a nucleic acid encoding a reprogramming factor using a CRISPR/Cas9 system containing a vector including gRNA 1 represented by SEQ ID NO: 1 and gRNA 2 represented by SEQ ID NO: 2.

Accordingly, an aspect of the present invention pertains to a method of producing induced pluripotent stem cells including inserting a nucleic acid encoding a reprogramming factor into CASH-1 (CRISPR/Cas9-accessible safe harbor-1) of somatic cells using a CRISPR/Cas system, in which the CRISPR/Cas system contains a delivery vehicle including nucleic acids encoding a gRNA (guide RNA) and a Cas protein, and the gRNA includes gRNA 1 represented by SEQ ID NO: 1 and gRNA 2 represented by SEQ ID NO: 2.

In the present invention, the CRISPR/Cas9 system may be a double-nicking CRISPR/Cas9 system with paired gRNA 1 and gRNA 2. A typical side effect occurring upon inserting a target gene into cells is an off-target effect. In order to reduce this error, a double-nicking CRISPR/Cas9 system including gRNA 1 and gRNA 2 was used.

In the present invention, CASH-1 may correspond to position 188,083,272 of chromosome 1 of somatic cells.

As used herein, the term “CASH-1” refers to CRISPR/Cas9-accessible safe harbor-1, and corresponds to position 188,083,272 of chromosome 1. The criteria for being a safe GSH are: (1) a >50 kbp distance from the 5′ end of a gene, (2) a >300 kbp distance from any cancer-associated gene, (3) a >300 kbp distance from any microRNA gene, and (4) locations outside a superconserved sequence and a gene transcription unit (Papapetrou, E. P. et al. Nature Biotechnology 29, 73 (2011)). CASH-1 may serve as an alternate GSH for successful functional expression of integrated transgene(s) because it satisfies all three of the above criteria as a GSH. On the other hand, GSHs such as AAVS1, CCR5, and hROSA26, as described above, have limitations such as effects on nearby genes, high risk of infection, low expression of integrated transgenes, and location in gene-rich regions.

In the present invention, the CRISPR/Cas system may include at least one delivery vehicle including nucleic acids encoding gRNA and a Cas protein.

Regarding the delivery vehicle, nucleic acids encoding a plurality of gRNAs and a Cas protein may be located on the same or different delivery vehicles. These delivery vehicles may be viral delivery vehicles simultaneously, a viral delivery vehicle and a non-viral delivery vehicle respectively, or non-viral delivery vehicles simultaneously.

In the present invention, the delivery vehicle may be at least one selected from the group consisting of vectors, ribonucleoprotein (RNP) complexes, carriers, and exosomes.

In the present invention, the nucleic acids encoding the gRNA and the Cas protein may be contained in the same or different delivery vehicles.

In the present invention, the nucleic acids encoding the gRNA and the Cas protein may be contained in the same or different vectors.

A vector, one of the delivery vehicles, may be designed for expression of nucleic acid transcripts, proteins, or enzymes of the Cas protein as well as the plurality of gRNAs in prokaryotic or eukaryotic cells. Respective DNA sequences encoding the gRNA sequence and the Cas protein may be separately placed on different vectors and delivered. Alternatively, the DNA sequences encoding the gRNA sequence and the Cas protein may be placed on the same vector and delivered simultaneously through one vector.

In the present invention, the vector may be a viral vector selected from the group consisting of an adeno-associated viral vector (AAV), an adenoviral vector (AdV), a lentiviral vector (LV), and a retroviral vector (RV); or may be an episomal vector including a viral replicon.

In the present invention, the episomal vector may include the origin of replication (Ori) of Simian virus 40 (SV40), bovine papilloma virus (BPV), or Epstein-Barr nuclear antigen (EBV).

Various methods of inserting genes into cells have been developed with recent advances in gene therapy. Also, thorough research is ongoing into toxicity for delivery of genes that are inserted harmlessly in the human body in the insertion method using a viral vector among various methods of inserting genes. Such research may also be achieved by reducing the number of enzymes inserted into cells. Upon insertion using the RNP complex, the dose of the RNP complex may be minimized, or the dose of the vector may be minimized when the vector is used for transformation.

Moreover, the gene may be inserted through a method of delivering the RNP complex using nanoparticles in addition to viral delivery. The use of nanoparticles is capable of minimizing side effects that may be caused in wild-type cells by linking a peptide that specifically binds to cancer cells to the outside of the nanoparticles. An RPARPAR peptide or the like that specifically targets cancer cells may be used (Sugahara et al., Cancer Cell (2009) 16:510-520).

The delivery vehicle may be, for example, a viral delivery vehicle. The viral delivery vehicle may include a viral vector. Examples of the viral vector may include, but are not limited to, an adeno-associated viral vector (AAV), an adenoviral vector (AdV), a lentiviral vector (LV), and a retroviral vector (RV) (Acta Biochim. Pol. 2005, 52(2): 285-291, and Biomolecules. 2020 Jun, 10(6): 839).

Regarding the AAV vector, International Patent Publication No. WO2016/176191 discloses a dual vector system for treating genetic diseases. The vector system is based on the AAV vector, and may include an AAV vector including a Cas9 gene and an AAV vector including at least one sgRNA. Here, with regard to the inclusion ratio of the AAV vector including the Cas9 gene and the AAV vector including at least one sgRNA, the AAV vector including the sgRNA may exceed the AAV vector including the Cas9 gene. For example, the ratio of the AAV vector including the Cas9 gene to the AAV vector including at least one sgRNA may be about 1:3 to about 1:100, particularly about 1:10. It is described that the ratio of a nuclease (e.g. Cas9 or Cpf) to a donor template may be maintained even when the nuclease is additionally or alternatively supplied by a source other than the AAV vector, which may be applied to the present invention.

Regarding the AdV vector, it has been reported that CRISPR/Cas9 may be delivered using HCAdV (high-capacity adenoviral vector), which is a third-generation AdV (Nature Scientific Reports volume 7, Article number: 17113 (2017)). Unlike the AAV, all components of the CRISPR/Cas9 system may be contained in a single vector. Constitutive or inducible Cas9 may be expressed and multiple gRNAs may be contained. CRISPR/Cas9-HCAdV targeting HPV 18 (human papillomavirus 18) oncogene E6, DMD (dystrophin gene causing Duchenne muscular dystrophy), and CCR5 (HIV co-receptor C—C chemokine receptor type 5) may be constructed, a CRISPR/Cas9-HCAdV expression unit may be delivered to the target, and a DNA DSB may be induced at the desired target site. The above may be applied to the composition according to the present invention and delivery thereof and incorporated by reference.

As described in WO2017/223330, also, CRISPR/Cas may be delivered using the genome of single-stranded RNA (ssRNA) virus. LV and AAV have been used for cell transformation in ex-vivo and in-vivo forms, but viral DNA-based replication carries the risk of undesirable genotoxicity or carcinogenesis in the host genome, suggesting a different form of ssRNA viral vector than before.

Mononegavirales including a linearly single-stranded non-infectious RNA chain with negative polarity may be exemplified, which may be replicated by producing 5-10 different mRNAs through polar sequential transcription and synthesizing the complete antigenome. Examples of Mononegavirales may include Bornaviridae, Filoviridae, Nyamiviridae, Paramyxodiridae, and Rhabdoviridae. Bornaviridae may include bornavirus. Filovindae may include cuevavirus, ebolavirus, and marburgvirus. Nyamiviridae may include nyavirus. Paramyxoviridae may include aquaparamyxovirus, avulavirus, feravirus, heniparvirus, morbillivirus, respirovirus, rubulavirus, pneumovirus, metapneumovirus, and sendai virus. Rhabdoviridae may include alemndravirus, baiavirus, curiovirus, cytorhabdovirus, dichorhavirus, ephemerovirus, hapavirus, ledantevirus, lyssavirus, novirhabdovirus, nuclearhabdovirus, perhabdovirus, sawgravirus, sigmavirus, sprivivirus, tibrovirus, tupavirus, and vesiculovirus. In particular, a CRISPR/Cas delivery system that is constructed using Sendai virus is disclosed in WO2017/223330. As such, by applying a self-cleaving ribozyme to gRNA of the CRISPR/Cas system, gRNA may be effectively cleaved/released when introduced into the host cell. Thereby, RNA transfection efficiency was confirmed to increase. The above may be applied to the composition according to the present invention and delivery thereof and incorporated by reference.

The delivery vehicle may be, for example, a non-viral delivery vehicle. The non-viral delivery vehicle may be exemplified by an episomal vector including a viral replicon. The non-viral delivery vehicle enables delivery in the form of mRNA or delivery by an RNP (ribonucleoprotein) or a carrier. Delivery in the form of mRNA may include synthetic modified mRNA or self-replicating RNA (srRNA). The carrier may include nanoparticles, cell penetrating peptides (CPPs), or polymers (Nature Reviews Genetics volume 15, pages 541-555 (2014)). The non-viral delivery vehicle may include delivery via exosomes.

Regarding the RNP, a pre-formed RNP may be introduced into cells to initiate targeted genomic DNA editing. This delivery using the RNP has advantages of reducing off-target effects compared to delivery using the vector and enabling direct delivery of the Cas protein and gRNA into the cell nucleus (Scientific Reports volume 8, Article number: 14355 (2018)).

In order to form the RNP (ribonucleoprotein), the Cas protein and gRNA may be individually synthesized, purified, and produced in an RNP form. In some cases, a CL7-tagged Cas protein and a cleavage factor RNA may be directly expressed through a plasmid, after which a self-assembled RNP may be purified and produced (Curr. Protoc. Mol. Biol. 2017 Oct. 2,120:31.10.1-31.10.19).

International Patent Publication No. WO2019/089884 discloses a genome editing system including gRNA and an RNA-guided nuclease as well as a vector including gRNA and an RNA-guided nuclease-encoding polynucleotide in order to modify TGFBR2 gene expression in cells. In accordance with WO2019/089884, gRNA and the RNA-guided nuclease may be provided in the form of a ribonucleoprotein (RNP) complex, two or more different ribonucleoprotein (RNP) complexes may be provided, and gRNAs having different target domains may be provided.

Regarding delivery through a carrier, mRNA encoding the Cas protein and/or gRNA may be individually constructed or provided in the form of a ribonucleoprotein (RNP), which may then be delivered through a carrier delivery system. Examples of the carrier may include cell penetrating peptides (CPPs), nanoparticles, zeolitic imidazole frameworks (ZIFs), RNA aptamer-streptavidin, and polymers (Biomaterials. July 2018, 171: 207-218).

Regarding the nanoparticles, the composition according to the present invention may be delivered via polymer nanoparticles, metal nanoparticles, metal/inorganic nanoparticles, or lipid nanoparticles.

The polymer nanoparticles may be exemplified by DNA nanoclews or thread-like DNA nanoparticles synthesized by rolling circle amplification. DNA nanoclews or thread-like DNA nanoparticles are loaded with Cas9/sgRNA and coated with PEI to improve endosomal escape capacity. These complexes bind to the cell membrane, are internalized, and then move to the nucleus via endosomal escape, allowing simultaneous delivery of the Cas9 protein and the single guide RNA (Angew Chem. Int. Ed. Engl. 2015 Oct. 5; 54(41) 12029-12033). The above may be applied to delivery of the composition according to the present invention and incorporated by reference.

In another embodiment, cationic and anionic acrylate monomers may be used to coat the RNP of the Cas9 protein and the single guide RNA through electrostatic interactions. Imidazole-containing monomers, GSH-degradable crosslinkers, acrylate mPEG, and acrylate PEG conjugated ligands may be linked and attached to the RNP surface. Through in-situ free-radical polymerization, glutathione (GSH)-cleavable nanocapsules may be formed around RNPs, and the nanocapsules may be crosslinked with N,N′-bis(acryloyl)cystamine serving as a GSH-cleavable linker and degraded in the cytoplasm, allowing RNP degradation in the cytoplasm (Nature Nanotechnology volume 14, pages 974-980 (2019)). The above may be applied to delivery of the composition according to the present invention and incorporated by reference.

Regarding the metal nanoparticles, a Cas9 ribonucleoprotein and donor DNA may be delivered to cells by linking gold particles to DNA and forming a complex with a cationic endosomal disruptive polymer (Nature Biomedical Engineering volume 1, pages 889-901 (2017)). Examples of the cationic endosomal disruptive polymer may include polyethylene imine, poly(arginine), poly(lysine), poly(histidine), poly-[2-{(2-aminoethyl)amino}-ethyl-aspartamide (pAsp(DET)), block copolymer of poly(ethylene glycol) (PEG) and poly(arginine), block copolymer of PEG and poly(lysine), and block copolymer of PEG and poly{N-[N-(2-aminoethyl)-2-aminoethyl]aspartamide} (PEG-pAsp(DET)).

In another embodiment, magnetic nanoparticles and a recombinant baculoviral vector may be complexed and activated locally under a magnetic field to allow CRISPR-Cas9-mediated gene editing to occur (Nature Biomedical Engineering volume 3, pages 126-136 (2019)). The above may be applied to delivery of the composition according to the present invention and incorporated by reference.

In still another embodiment, a photolabile semiconductor polymer nanotransducer (pSPN) may be used as a gene vector that delivers a CRISPR/Cas9 plasmid into cells and may be useful as a photomodulator for remotely activating gene editing. pSPN may include a single oxygen (102) generating backbone grafted with a polyethylenimine brush via a 1O2 cleavable linker. Cleavage of the gene vector in pSPN is spontaneously induced by near-infrared (NIR) light irradiation and the CRISPR/Cas9 plasmid is released, initiating gene editing (Angew Chem. Int. Ed. Engl. 2019 Dec. 9; 58 (50) 18197-18201). The above may be applied to delivery of the composition according to the present invention and incorporated by reference.

Regarding the metal/inorganic nanoparticles, zeolitic imidazolate framework-8 (ZIF-8) may be used to encapsulate CRISPR/Cas9 and also to alter the expression of target genes of interest through efficient endosomal escape (Advanced Therapeutics Volume 2, Issue 4 April 2019). The above may be applied to delivery of the composition according to the present invention and incorporated by reference.

Through the ZIF (zeolitic imidazole framework), the negatively charged CRISPR RNP may be encapsulated with the positively charged nanoscale ZIF. The endosomal escape of RNP may be enhanced by ZIF (J. Am. Chem. Soc. 2018, 140:143-146). The above may be applied to the composition according to the present invention and delivery thereof.

As RNA aptamer-streptavidin, S1mplex (modular RNA aptamer-streptavidin strategy) may be used. In this regard, a 60-nucleotide S1m aptamer that interacts non-covalently with streptavidin may be added to the 3′ end of sgRNAs. Then, a complex thereof with streptavidin and Cas9 protein may be formed, and may be delivered into cells as S1mplex. In some cases, biotinylated single-stranded oligodeoxynucleotide (ssODN) having high affinity for streptavidin may be incorporated as a nucleic acid donor template. It was confirmed that nucleic acid may be modified precisely using the complex thus configured. Increased gene editing efficiency may be exhibited compared to standard sgRNA RNPs (Nat. Commun. 2017, 8:1711). The above may be applied to the composition of the present invention and delivery thereof.

DNA or nucleic acid constituting the negatively charged CRISPR/Cas9 may be coupled with cationic materials to form nanoparticles, which may penetrate cells through receptor-mediated endocytosis or phagocytosis. For example, U.S. Pat. No. 10,682,410 discloses a technique for delivering a Cas protein to cells using a negatively charged Cas9 protein-RNA formed through binding between positively charged Cas9 protein and strongly negatively charged guide RNA, a supercharged protein (e.g. positively supercharged protein), a cationic polymer, or a cationic lipid, which may be applied to the present invention. The supercharged protein, cationic polymer, or cationic lipid may be introduced into cells by contacting the Cas protein. For delivery to the target cells, the Cas9 protein provides a negative charge and is coupled with a supercharged GFP such as +36 GFP or −30 GFP, as a supercharged fluorescent protein. Endosomes may be advantageously disrupted by the supercharged protein, increasing endosomal escape. The above may be applied to the composition of the present invention and delivery thereof.

Regarding the cationic polymer, a Cas9:gRNA complex may bind to the cationic polymer. Examples of the cationic polymer may include polyallylamine (PAH); polyethyleneimine (PEI); poly(L-lysine) (PLL); poly(L-arginine) (PLA); polyvinylamine homopolymers or copolymers; poly(vinylbenzyl-tri-C1-C4-alkylammonium salts); polymers of aliphatic or alicyclic dihalides and aliphatic N,N,N′,N′-tetra-C1-C4-alkyl-alkylenediamines; poly(vinylpyridine) or poly(vinylpyridinium salt); poly(N,N-diallyl-N,N-di-C1-C4-alkyl-ammonium halide); homopolymers or copolymers of quaternized di-C1-C4-alkyl-aminoethyl acrylates or methacrylates; POLYQUAD™; polyaminoamide, and the like.

Regarding the polymer, a flexible dendritic polymer and a CRISPR Cas system may be delivered into cells (Chem. Sci. 2017, 8:2923-2930). Also, the CRISPR Cas system may be delivered into cells, for example via a hybrid polymeric microcarrier made of polypeptide and polysaccharide (Nanomedicine. 2018, 14:97-108). Furthermore, the CRISPR Cas system may be delivered into cells, for example via a GO-PEG-PEI (graphene oxide-poly(ethylene glycol)-polyethylenimine) nanocarrier (Nanoscale. 2018, 10:1063-1071).

Regarding the lipid nanoparticles, the CRISPR Cas system may be delivered using liposomes as the carrier. Liposomes are spherical vesicular structures composed of a unilamellar or multilamellar lipid bilayer surrounding an inner aqueous compartment and a relatively impermeable outer lipophilic phospholipid bilayer. Liposomal formulations may primarily contain natural phospholipids and lipids such as 1,2-distearoyl-sn-glycero-3-phosphatidylcholine (DSPC), sphingomyelin, phosphatidylcholine, or monosialoganglioside. In some cases, cholesterol or 1,2-dioleoyl-sn-glycero-3-phosphoethanolamine (DOPE) may be added to the lipid membrane in order to resolve instability in plasma. The addition of cholesterol reduces the rapid release of encapsulated bioactive compounds into plasma or 1,2-dioleoyl-sn-glycero-3-phosphoethanolamine (DOPE) increases stability (Semple et al., Nature Biotechnology, Volume 28 Number 2 February 2010, pp. 172-177).

In the context of delivery via exosomes, “exosomes” are cell-derived small (20-300 nm diameter, more preferably 40-200 nm diameter) vesicles that contain a membrane surrounding an interior space, and refer to vesicles produced from the cells either by direct plasma membrane budding or by fusion of late endosomes with the plasma membrane. Generally, production of exosomes does not result in destruction of producer cells. An exosome includes a lipid or fatty acid and a polypeptide, optionally a payload (e.g. a therapeutic agent), a receiver (e.g. a targeting moiety), a polynucleotide (e.g. nucleic acid, RNA, or DNA), a sugar (e.g. monosaccharide, polysaccharide, or glycan), or other molecules. Exosomes are derived from the producer cells and may be isolated from the producer cells based on the size, density, or biochemical parameters thereof or a combination thereof.

The CRISPR-Cas9 system may be delivered via functionalized exosomes (Biomater Sci. 2020 May 19;8(10):2966-2976). GFP and GFP nanobody may be coupled with CD63, which is an exosome membrane protein, and Cas9 protein may be fused to exosomes. Thereby, it was confirmed that the target gene may be efficiently edited in the recipient cell. Such exosomes enable delivery of the composition according to the present invention and are incorporated by reference.

An example of the delivery vehicle useful in the present invention may include a vector. The term “vector” refers to a nucleic acid molecule capable of transporting another nucleic acid to which it has been linked as a nucleic acid molecule. Examples thereof may include nucleic acid molecules that are single-stranded, double-stranded, or partially double-stranded; nucleic acid molecules including one or more free ends or non-free ends (e.g. circular); nucleic acid molecules including DNA, RNA, or both; and a variety of other polynucleotides known in the art.

For example, the vector is a “plasmid,” which refers to a circular double-stranded DNA loop into which additional DNA segments may be inserted using standard molecular cloning techniques.

For viral vectors, virus-derived DNA or RNA sequences are included in the vector for packaging into viruses (e.g. retroviruses, replication defective retroviruses, adenoviruses, replication defective adenoviruses, and adeno-associated viruses). Viral vectors include polynucleotides carried by viruses for transfection into host cells.

In some cases, the vector is capable of autonomous replication in a host cell into which it is introduced (e.g. bacterial vectors having bacterial replicating Ori and episomal mammalian vectors). Other vectors (e.g. non-episomal mammalian vectors), upon introduction into a host cell, are integrated into the genome of a host cell and are thereby replicated along with the host genome.

Certain vectors are capable of directing the expression of genes to which they are operably linked. Such vectors are referred to herein as “expression vectors”. Common expression vectors useful in recombinant DNA technology are often in the form of plasmids.

A recombinant expression vector may include a nucleic acid in a form suitable for expression of the nucleic acid in a host cell, which means to include at least one regulatory element that is selected on a host cell basis so that the recombinant expression vector is used for expression, namely is operably-linked to the nucleic acid sequence to be expressed.

In some cases, the vector includes at least one pol III promoter, at least one pol II promoter, at least one pol I promoter, or a combination thereof. Examples of the pol III promoter include, but are not limited to, U6 and H1 promoters. Examples of the pol II promoter include, but are not limited to, retroviral Rous Sarcoma Virus (RSV) LTR promoter (optionally with RSV enhancer), cytomegalovirus (CMV) promoter (optionally with CMV enhancer) (e.g. Boshart et al. (1985) Cell 41:521-530), SV40 promoter, dihydrofolate reductase promoter, p-actin promoter, phosphoglycerol kinase (PGK) promoter, and EF1α promoter.

The vector may be introduced and propagated in prokaryotes. In some embodiments, prokaryotes may be used to amplify copies of vectors to be introduced into eukaryotic cells or as intermediate vectors in the production of vectors to be introduced into eukaryotic cells (e.g. amplification of a plasmid as part of a viral vector packaging system). Prokaryotes may be used to amplify copies of the vector, express at least one nucleic acid, and provide a source of at least one protein for delivery into a host cell or host organism. Protein expression in prokaryotes may be performed in E. coli with vectors containing constitutive or inducible promoters.

The vector may be delivered in vivo or into cells through electroporation, lipofection, viral vectors, nanoparticles, PTD (protein translocation domain) fusion protein methods, etc.

In the present invention, the vector including the nucleic acids encoding the gRNA and the Cas protein may be pX330-U6-Chimeric_BB-CBh-hSpCas9 or pX335-U6-Chimeric_BB-CBh-hSpCas9n(D10A).

In the present invention, the Cas protein may be a Cas3, Cas9, Cpf1, Cas6, C2c12, or C2c2 protein.

In the present invention, the Cas protein may be derived from the genus of microorganisms containing an ortholog of the Cas protein selected from the group consisting of Corynebacter, Sutterella, Legionella, Treponema, Filifactor, Eubacterium, Streptococcus, Lactobacillus, Mycoplasma, Bacteroides, Flaviivola, Flavobacterium, Azospirillum, Gluconacetobacter, Neisseria, Roseburia, Parvibaculum, Staphylococcus, Nitratifractor, Corynebacterium, and Campylobacter, and may be isolated therefrom or recombinant.

In the present invention, the Cas9 protein may be wild-type Cas9, deactivated Cas9 (dCas9), or Cas9 nickase.

In the present invention, the Cas9 nickase may be SpnCas9 (SpCas9-derived nickase). The SpnCas9 is a SpCas9-derived nickase converted by catalytically deactivating one of the domains of the nuclease SpCas9.

In the present invention, the nucleic acid encoding the reprogramming factor may be inserted using a vector including the nucleic acid encoding the reprogramming factor.

In the present invention, the reprogramming factor may be i) delivered to somatic cells as a proteinaceous factor itself through, when a material is a proteinaceous factor, a method using a protein delivery reagent, a method using a protein delivery domain (PTD)- or cell penetrating peptide (CPP)-fusion protein, or a known protein delivery method such as microinjection or electroporation, or may be ii) included in a vector and delivered in the form of a nucleic acid encoding a reprogramming factor.

In the present invention, the vector including the nucleic acid encoding the reprogramming factor may be a viral vector or a non-viral vector.

The viral vector may be selected from the group consisting of an adeno-associated viral vector (AAV), an adenoviral vector (AdV), a lentiviral vector (LV), and a retroviral vector (RV). The non-viral vector is an expression vector capable of autonomous replication outside the chromosome, and the non-viral expression vector is preferably not integrated into a chromosome, and may be a plasmid vector or an episomal vector.

Examples of the plasmid vector may include, but are not limited to, plasmids derived from Escherichia coli (ColE-based plasmids such as pBR322, pUC18, pUC19, pUC118, pUC119, pBluescript, etc.), plasmids derived from Actinomyces (pIJ486, etc.), plasmids derived from Bacillus subtilis (e.g. pUB110, pSH19 and others), yeast-derived plasmids (YEp13, YEp 24, Ycp50, etc.), artificial plasmid vectors, and the like.

In the present invention, the vector including the nucleic acid encoding the reprogramming factor may include a single polycistronic cassette.

In the present invention, the vector including the nucleic acid encoding the reprogramming factor may be pUC57-Amp.

In the present invention, the reprogramming factor may be any protein factor (group), for example, at least one factor selected from the group consisting of Oct family, Klf family, Sox family, Myc family, Lin family, Glis family, and Nanog genes, so long as iPS cells may be induced by introducing a nucleic acid encoding the same into somatic cells (Science, 318, pp. 1917-1920, 2007; WO 2007/69666).

Preferably, the reprogramming factor is at least one selected from the group consisting of OCT4, SOX2, KLF4, c-MYC, NANOG, SSEA4, and TRA1-60, more preferably OCT4, SOX2, and KLF4 (Takahashi, K. and Yamanaka, S., Cell, 126: 663-676 (2006)), but the present invention is not limited thereto.

In the present invention, expression of the reprogramming factor may be controlled with doxycycline (DOX). Controlled expression of the reprogramming factor is capable of safely producing iPSCs. Constitutive expression of reprogramming genes such as OCT4 and SOX2 may promote differentiation of embryonic stem cells, and thereby may affect clinical applications such as disease modeling (Niwa H., Miyazaki, J.-i. & Smith, A. G. Nature Genetics 24, 372-376 (2000); Kopp, J. L., et al., Stem Cells 26, 903-911 (2008)). Therefore, iPSCs according to the present invention were produced by controlling expression of the reprogramming factor with DOX.

In the present invention, the molar ratio of the vector including the nucleic acids encoding the gRNA and the Cas protein to the vector including the nucleic acid encoding the reprogramming factor may be 1:1-1:3.

In the present invention, the vector including the nucleic acid encoding the reprogramming factor may be circular (or uncut) or linear (or cut), preferably linear (or cut), but the present invention is limited thereto.

In the present invention, the somatic cells may be mouse fibroblasts, human fibroblasts (HDFs), human kidney cells (HEK293T), human keratinocytes, or human peripheral blood cells, preferably human fibroblasts (HDFs) or human kidney cells (HEK293T), but are not limited thereto.

As used herein, the term “induced pluripotent stem cell” refers to a cell induced to have pluripotent differentiation potential through an artificial dedifferentiation process from differentiated cells, and is also called a dedifferentiated induced pluripotent stem cell. Induced pluripotent stem cells have almost the same characteristics as embryonic stem cells, and particularly have similar cell appearance and gene and protein expression patterns, have pluripotency in vitro and in vivo, form teratomas, and enable genetic germline transmission. The induced pluripotent stem cells according to the present invention include dedifferentiated induced pluripotent stem cells derived from all mammals such as humans, monkeys, pigs, horses, cattle, sheep, dogs, cats, mice, rabbits, and the like, and are particularly human-derived induced pluripotent stem cells, most particularly induced pluripotent stem cells derived from FXS patients.

As used herein, the term “dedifferentiation” refers to an epigenetic retrograde process that returns existing differentiated cells to an undifferentiated state to enable the formation of new differentiated tissue, which is also called a “reprogramming” process, and is based on the reversibility of epigenetic changes in the cell genome. For the purpose of the present invention, “dedifferentiation” includes any process of returning differentiated cells having a differentiation potential of 0% to less than 100% to an undifferentiated state, and for example, a process of undifferentiating differentiated cells having a differentiation potential of 0% into differentiated cells having a differentiation potential of 1% may also be included.

As used herein, the term “CRISPR/Cas system” refers to a gene editing tool through guide RNA, in which, using a bacterially derived endonuclease Cas (or mutant nickase) and guide RNA, a double (or single)-strand break may be introduced at a specific site in the genome by matching sequences between guide RNA and genomic DNA.

The “CRISPR/Cas9 system” is a genome editing tool called CRISPR (clustered regularly interspaced short palindromic repeat) gene scissors, which includes guide RNA (gRNA) and Cas9 (CRISPR associated protein 9: RNA-guided DNA endonuclease enzyme). Guide RNA includes crRNA (CRISPR RNA) and tracrRNA (trans-activating crRNA), and binds to Cas9 and guides the same to the desired genomic sequence through base pairing to the target sequence, resulting in double-strand breaks (DSBs). The only criterion for defining a target sequence is the presence or absence of PAM (protospacer adjacent motif). The sequence of PAM may vary depending on the Cas protein recognizing the same.

The use of the CRISPR/Cas9 system enables knock-in capable of expressing a specific gene or knock-out capable of suppressing the function of a specific gene by introducing plasmid DNA into cells or animals. A CRISPR/Cas9 system using single gRNA may tolerate specific mismatches to the DNA target, promoting unwanted off-target mutagenesis, whereas a double-nicking CRISPR/Cas9 system using paired gRNAs is suitable for genome editing with increased specificity and minimized off-target mutagenesis by expanding the number of bases specifically recognized at the target site (F. Ann Ran et al., Double nicking by RNA-guided CRISPR Cas9 for enhanced genome editing specificity, Cell. 2013 September 12; 154(6): 1380-1389).

The Cas9 nuclease uses HNH and RuvC nuclease domains to generate blunt-end DSB, which is mainly repaired by an error-prone DNA repair pathway—non-homologous end joining—in the absence of a repair template by creating indel mutations. In the presence of Cas9, the percentage of indels obtained by a given gRNA directly determines the genome editing ability thereof. Catalytic deactivation of any one of the SpCas9 nuclease domains converts the same to a DNA “nickase”, which produces a single-strand break at the target site. Since SpnCas9 has very good specificity for transgene integration at the target site compared to wild-type SpCas9 (Mali, P. et al. Nature Biotechnology 31, 833 (2013)), for integration of the reprogramming cassette in CASH-1 in the present invention, a double-nicking approach using gRNAs g1 and g2 in the presence of SpCas9-derived nickase (SpnCas9) is used.

As used herein, the term “transfection” refers to introducing DNA into a host so that the DNA becomes replicable as an extrachromosomal factor or by completion of chromosomal integration. Transfection may include any method of introducing a nucleic acid molecule into an organism, cell, tissue, or organ, and may be performed by selecting a suitable standard technique depending on the type of host cell as known in the art.

The vector of the present invention preferably includes at least one selection marker. In the present invention, the selection marker is used to select cells transformed with a gene-targeting vector, and examples thereof may include markers conferring selectable phenotypes such as drug resistance, auxotrophy, resistance to cytotoxic agents, or expression of surface proteins. There are positive selection markers and negative selection markers.

As used herein, the term “donor vector” refers to a vector including a nucleic acid encoding a reprogramming factor.

As used herein, the term “cassette” may include at least one polycistronic transcription unit. The polycistronic unit may include different combinations of operably linked coding regions, for example (i) at least two reprogramming genes (e.g. Sox-Oct, c-Myc-Klf, or Nanog-Lin28); or (ii) a reprogramming gene linked to a selectable or selection marker.

As used herein, the term “polycistronic” gene refers to a gene that contains two or more coding sequences (i.e. cistrons) and encodes more than one protein. Polycistronic mRNA may include any element known in the art to allow translation of two or more genes from the same mRNA molecule, including but not limited to IRES element, T2A element, P2A element, E2A element, and F2A element.

Another aspect of the present invention pertains to induced pluripotent stem cells produced through the method described above.

A better understanding of the present invention may be obtained through the following examples. These examples are merely set forth to illustrate the present invention, and are not to be construed as limiting the scope of the present invention, as will be apparent to those skilled in the art.

### EXAMPLES

### Example 1-1: Design and Confirmation of Guide RNAs

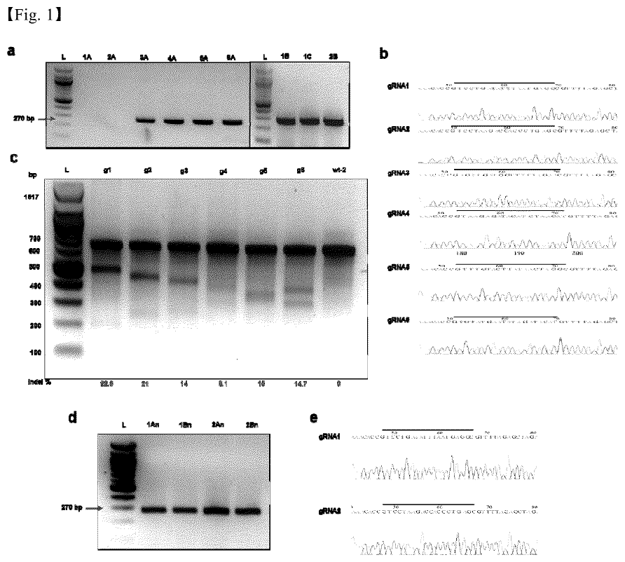

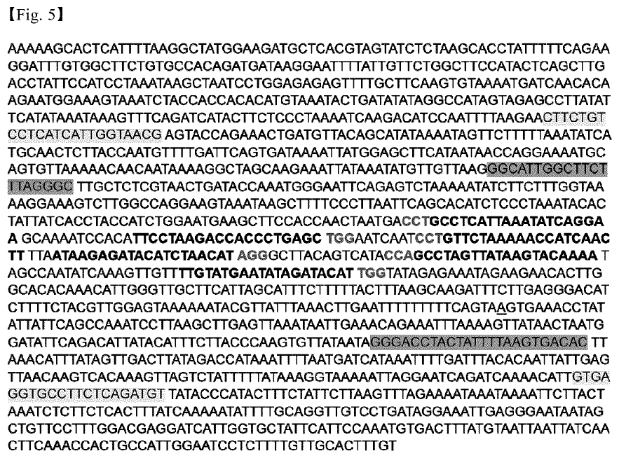

Any one of the aforementioned GSHs (CASH-1; chromosome 1: position 188,083,272) was randomly selected to confirm accessibility thereof to a CRISPR/Cas9 genome editing tool. To this end, six target sites throughout GSH were selected based on the 5′-NGG-3′ PAM sequence, which is recognized by Streptococcus pyogenes-derived Cas9 (SpCas9) (FIG. 5). A gRNA oligonucleotide specific to each site was designed, annealed, and cloned into the BbsI-digested pX330-U6-Chimeric_BB-CBh-hSpCas9 vector. Most clones obtained after antibiotic selection were colony PCR positive except for a few clones such as 1A and 2A, and were identified as 1B, 1C, and 2B in the next round (FIG. 1a). All clones were further confirmed by Sanger sequencing (FIG. 1B, Table 1). The designed construct constitutively expresses the cloned gRNA and human codon-optimized SpCas9 enzyme upon transfection into human cells. The ability of each of six gRNAs to modify the target site was determined by transfecting HEK293T cells with the relevant gRNA vector along with a negative control vector encoding only hSpCas9 (without any gRNA). The wild-type Cas9 enzyme has two endonuclease domains capable of cleaving both strands of target DNA that are later repaired by the error-prone DNA repair pathway “non-homologous end joining (NHEJ)”. This creates mismatches (insertions/deletions) in the repaired DNA that may be detected in vitro through T7E1 endonuclease assay. T7 endonuclease I assay was performed using amplicons and the percentage of insertions/deletions (indels) for each gRNA was calculated. Indel percentages calculated using ImageJ software were 22.6%, 21%, 14%, 8.1%, 15%, and 14.7% for g1, g2, g3, g4, g5, and g6, respectively (FIG. 1c). The indel percentage indicates the ability of a given gRNA to promote gene editing at the target site thereof. Thus, g1 and g2 are the most efficient gRNAs at almost the same levels of 22.6% and 21%, respectively. Then, g1 and g2 were annealed and cloned into the BbsI-digested pX335-U6-Chimeric BB-CBh-hSpCas9n(D10A) vector. Two randomly selected clones for g1 (SEQ ID NO: 1) and g2 (SEQ ID NO: 2) were confirmed by colony PCR (FIG. 1d) and Sanger sequencing (FIG. 1e).

TABLE 1
gRNA sequences

| gRNA | Sequence | SEQ ID NO: |
|---|---|---|
| gRNA 1 | GTCCTGATATTTAATGAGGC | 1 |
| gRNA 2 | GTCCTAAGACCACCCTGAGC | 2 |
| gRNA 3 | GAGTTGATGGTTTTTAGAAC | 3 |
| gRNA 4 | GTAAGAGATACATCTAACAT | 4 |
| gRNA 5 | GTTTGTACTTATAACTAGGC | 5 |
| gRNA 6 | GTGTATGAATATAGATACAT | 6 |

### Example 1-2: Design and Cloning of gRNAs

Six target sites were selected across the CASH-1 region, and 20-nucleotide-long gRNAs were designed based on the 5′-NGG-3′ PAM sequence. The relevant oligonucleotides were designed in a manner in which (i) each forward oligo starts with guanine and (ii) forward and reverse oligos have four extra nucleotides at the 5′ end, allowing the annealed gRNA to be complementary to the overhang of the digested target vector (Table 2).

TABLE 2
Target site-specific CRISPR RNA (crRNA) oligonucleotides
in CASH-1

| | | | | SEQ |
|---|---|---|---|---|
| | Target Site sequence | | | ID |
| Name | (5′-3′) | Direction | 5′-Sequence-3′ | NO: |
| g1 | CCTGCCTCATTAAATATCAGGAA | F | CACCGTCCTGATATTTAATGAGGC | 7 |
| | | R | AAACGCCTCATTAAATATCAGGAC | 8 |
| g2 | TTCCTAAGACCACCCTGAGCTGG | F | CACCGTCCTAAGACCACCCTGAGC | 9 |
| | | R | AAACGCTCAGGGTGGTCTTAGGAC | 10 |
| g3 | CCTGTTCTAAAAACCATCAACTT | F | CACCGAGTTGATGGTTTTTAGAAC | 11 |
| | | R | AAACGTTCTAAAAACCATCAACTC | 12 |
| g4 | ATAAGAGATACATCTAACATAGG | F | CACCGTAAGAGATACATCTAACAT | 13 |
| | | R | AAACATGTTAGATGTATCTCTTAC | 14 |
| g5 | CCAGCCTAGTTATAAGTACAAAA | F | CACCGTTTGTACTTATAACTAGGC | 15 |
| | | R | AAACGCCTAGTTATAAGTACAAAC | 16 |
| g6 | TTGTATGAATATAGATACATTGG | F | CACCGTGTATGAATATAGATACAT | 17 |
| | | R | AAACATGTATCTATATTCATACAC | 18 |

The vectors pX330-U6-Chimeric_BB-CBh-hSpCas9 (Addgene #42230) and pX335-U6-Chimeric_BB-CBh-hSpCas9n(D10A) (Addgene #42335) were digested with BbsI-HF (NEB #R3539) and treated with a calf intestinal alkaline phosphatase (NEB #M0290) according to the manufacturer's protocol. At a final concentration of 10 M, oligonucleotides were annealed in a mixture of ATP (1×final concentration; NEB), T4 PNK buffer (1×final concentration: NEB), and T4 PNK (polynucleotide kinase, 10 U; NEB). The annealing conditions in a thermocycler were adjusted as follows: 95° C. for 5 minutes followed by a −5° C./(3 minutes) ramp down to 65° C.; 63° C. for 3 minutes followed by a −3° C./(3 minutes) ramp down to 27° C., and final incubation at 25° C. for 10 minutes. The PNK-treated annealed oligonucleotides were diluted to a final concentration of 1 μM and ligated into the aforementioned digested vectors at 16° C. for 16 hours using a T4 DNA ligase enzyme (NEB #M0202). DH10B (Invitrogen) competent cells were transformed with constructs, spread onto ampicillin-containing (100 Hg/ml) 2XYT agar plates, and allowed to grow overnight at 37° C. One to two colonies were randomly selected for colony PCR analysis using a forward primer specific to the U6 promoter sequence (Table 3) and a reverse primer specific to the relevant cloned gRNA (Table 2).

TABLE 3
Oligonucleotides

| | | | | |
|---|---|---|---|---|
| | | | SEQ | Amplicon |
| Name | Direction | 5′-sequence-3′ | ID NO: | size (bp) |

Primers for colony PCR and sequencing

| | | | | |
|---|---|---|---|---|
| U6 promoter | F | GAGGGCCTATTTCCCATG | 19 | 270 |

Primers for T7E1 assay

| | | | | |
|---|---|---|---|---|
| 1^{st} primer set | F | CTTCTGTCCTCATCATTGGTAACG | 20 | 981 |
| | R | ACATCTGAGAAGGCACCTCAC | 21 | |
| 2^{nd} primer set | F | GGCATTGGCTTCTTTAGGGC | 22 | 628 |
| | R | GTGTCACTTAAAATAGTAGGTCCC | 23 | |

Primers for junction and donor PCR

| | | | | |
|---|---|---|---|---|
| 5′ Junction | Outer-FP | TCCAATAAAATAAGCGGGTGAC | 24 | 3081 |
| PCR | Donor-RP2 | GGCGCCCTGGTTTACATAAG | 25 | |
| 3′ Junction | Donor-FP6 | GTTCCCATCTCAAGGCACAC | 26 | 3126 |
| PCR | Outer-RP | TCCACCATTCTCCCACTGAC | 27 | |
| 3′ A (FC 1:3) | Donor-FP | TCAAGGTCAAAATCGTCAAGAGC | 28 | 8442 |
| | Outer-RP | TCCACCATTCTCCCACTGAC | 29 | |
| 3′ B (FC 1:3) | Donor-FP3 | CTTATGTAAACCAGGGCGCC | 30 | 6643 |
| | Outer-RP | TCCACCATTCTCCCACTGAC | 31 | |
| 3′ C (FC 1:3) | Donor-FP5 | CCAGCTCGCAGACCTACATG | 32 | 4758 |
| | Outer-RP | TCCACCATTCTCCCACTGAC | 33 | |
| Donor D (FC | Donor-FP | TCAAGGTCAAAATCGTCAAGAGC | 34 | 4004 |
| 1:3) | Donor-RP4 | CATGTGTGAGAGGGGCAGTG | 35 | |
| 5′ E (FUC | Outer-FP | TCCAATAAAATAAGCGGGTGAC | 36 | 4267 |
| 1:1) | Donor-RP3 | GTTTGAATGCATGGGAGAGC | 37 | |

Primers for RT-PCR

| | | | | |
|---|---|---|---|---|
| OCT4 | F | TTGGGCTCGAGAAGGATGTG | 38 | 91 |
| | R | TCCTCTCGTTGTGCATAGTCG | 39 | |
| SOX2 | F | GCCCTGCAGTACAACTCCAT | 40 | 85 |
| | R | TGCCCTGCTGCGAGTAGGA | 41 | |
| KLF4 | F | CGCCGCTCCATTACCAAGAG | 42 | 82 |
| | R | CACGATCGTCTTCCCCTCTT | 43 | |
| GAPDH | F | GTGGACCTGACCTGCCGTCT | 44 | 153 |
| | R | GGAGGAGTGGGTGTCGCTGT | 45 | |

Primers for qRT-PCR

| | | | | |
|---|---|---|---|---|
| Endo-OCT4 | F | GACAGGGGGAGGGGAGGAGCTAG | 46 | 142 |
| | R | CTTCCCTCCAACCAGTTGCCCCAAA | 47 | |
| Endo-SOX2 | F | TGGCGAACCATCTCTGTGGT | 48 | 111 |
| | R | CCAACGGTGTCAACCTGCAT | 49 | |
| Endo-KLF4 | F | ACAGTCTGTTATGCACTGTGGTTTCA | 50 | 84 |
| | R | CATTTGTTCTGCTTAAGGCATACTTGG | 51 | |
| c-MYC | F | CCAGCAGCGACTCTGAGGA | 52 | 75 |
| | R | GAGCCTGCCTCTTTTCCACAG | 53 | |
| NANOG | F | CAAGAAACAGAAGACCAGAACTGTG | 54 | 213 |
| | R | CCATTGCTATTCTTCGGCCAGTTG | 55 | |
| GAPDH | F | GTGGACCTGACCTGCCGTCT | 56 | 153 |
| | R | GGAGGAGTGGGTGTCGCTGT | 57 | |

Plasmid DNA was isolated from the correct clones using a PureLink HiPure Plasmid DNA Purification Kit (Invitrogen) according to the manufacturer's instructions and was processed for confirmation by Sanger sequencing (Macrogen, Korea).

### Example 2: Cell Culture and Reagents

HDF and HEK293T cells were maintained in DMEM (Gibco) supplemented with 10% FBS (Gibco), 1% penicillin/streptomycin solution (Gibco), and 100 μg/ml Normocin (InvivoGen). The cells were allowed to grow in a humidified atmosphere with 5% CO₂ at 37° C. and passaged three times a week. For cell cycle synchronization, cells at 70% confluency were maintained in serum-free medium for 48-72 hours. Thereafter, the medium was replaced with serum-containing medium for subsequent experiments.

### Example 3: T7E1 Endonuclease Assay

HEK293T cells were seeded at 10 5 cells per well in a 12-well plate and reverse-transfected with a 30-minute-preincubated mixture of 12 Hl of Lipofectamine 2000 (Invitrogen) and 6 μg of each gRNA vector (gRNA 1-6) or a control vector (vector not encoding gRNA), separately. After incubation for 10 hours, the medium was replaced with fresh complete medium, and the transfected cells were further incubated for 72 hours. Genomic DNA obtained from the cells in each treated well was separately isolated using a DNA Tissue Kit (Kurabo, Japan) according to the manufacturer's protocol. Genomic DNA flanking the gRNA target site was amplified by PCR with the first set of T7E1 primers. The PCR product was gel-purified using a PureLink™ Quick Gel Extraction Kit (Invitrogen) according to the manufacturer's protocol. The purified product was then amplified by PCR with the second set of T7E1 primers followed by gel-purification as described above. The primer sequences are listed in Table 3. The purified PCR product (1 μg) was mixed with 3.5 μl of 10×NEB 2 buffer (NEB) and double-distilled water was added to a final volume of 35 μl for formation of heteroduplexes via a reannealing process in a thermocycler (Applied Biosystems): 95° C. for 15 minutes, 25° C. for 1 minute, 95° C. for 10 minutes, 95-to-87° C. ramping at −2° C./5 seconds, 85° C. for 30 seconds, 85-to-77° C. ramping at −2° C./5 seconds, 75° C. for 30 seconds, 75-to-67° C. ramping at −2° C./5 seconds, 65° C. for 30 seconds, 65-to-57° C. ramping at −2° C./5 seconds, 55° C. for 30 seconds, 55-to-47° C. ramping at −2° C./5 seconds, 45° C. for 30 seconds, 45-to-37° C. ramping at −2° C./5 seconds, 35° C. for 30 seconds, 35-to-27° C. ramping at −2° C./5 seconds, and 25° C. for 30 seconds. After the reannealing process, the products were treated with 4.5 U of T7 endonuclease I (NEB # M0302) at 37° C. for 20 minutes and analyzed on a 2% ethidium bromide (Invitrogen)-stained agarose gel. Band intensities were quantified using ImageJ software (version 1.47V). The indel percentage for each target site was determined using the formula 100×(1−(1−(b+c)/(a+b+c))½), in which a is the intensity of the undigested PCR product, and b and c are the intensities of respective cleaved bands.

### Example 4-1: Knock-In of Reprogramming-Cassette

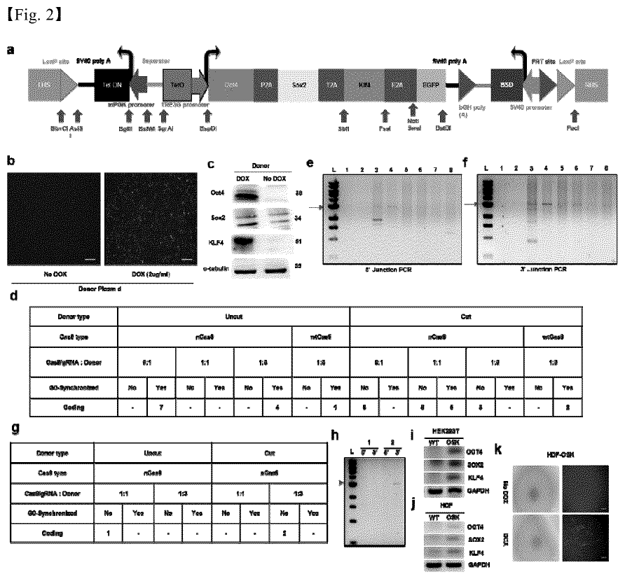

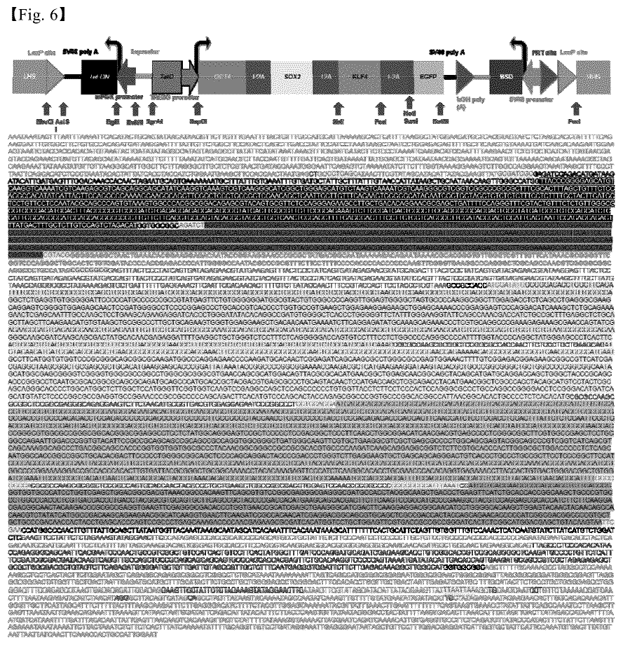

A DOX-inducible polycistronic reprogramming cassette was constructed through sequential arrangement of cDNAs of OCT4, SOX2, KLF4, and EGFP proteins, along with separation by sequence of cleavable 2A peptides (FIGS. 2a and 6). DOX-inducible expression of reprogramming factors in donor-transfected HEK293T cells was confirmed through fluorescence microscopy (FIG. 2b) and immunostaining for reprogramming factors (FIG. 2c). In order to improve specificity, a double-nicking CRISPR/Cas9 system with paired gRNAs g1 and g2 was used in the present invention.

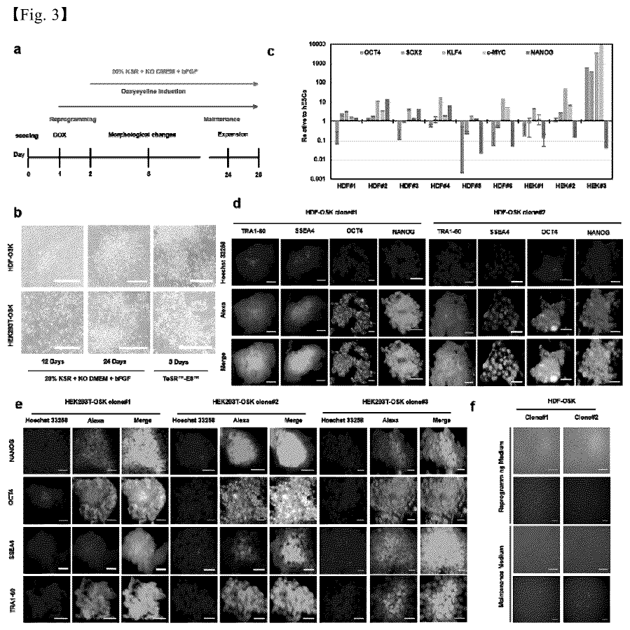

Also, knock-in of the donor cassette was optimized by considering factors affecting knock-in efficiency, such as the conformation of the donor, the ratio of the Cas9/gRNA vector to the donor vector, and the cell cycle phase of somatic cells. Marking schemes for knock-in optimization in HEK293T cells (FIG. 2d) and HDFs (FIG. 2g) are shown in respective drawings, including single gRNA (g1) with wild-type Cas9. The transfected cells that survived after antibiotic selection were assigned numerical codes for HEK293T cells (FIG. 2d) and HDFs (FIG. 2g), after which integrated donor cassettes were screened through 5′ junction PCR (FIGS. 2e) and 3′ junction PCR (FIG. 2f). Based on the results for HEK293T cells, linear (cut) donor cassettes with Cas9/gRNA and the donor at molar ratios of 1:1 and 1:3 yielded successful knock-in in unsynchronized cells. A circular (uncut) donor vector yielded knock-in of Cas9/gRNA and the donor at a molar ratio of 1:3 in cell cycle-synchronized cells (FIGS. 2e and 2f). For HDFs, it can be found that a linearized donor vector with Cas9/gRNA and the donor at a molar ratio of 1:3 is effective in unsynchronized cells (FIG. 2g). Surprisingly, HDF only tested positive in 3′ junction PCR (but not in 5′ junction PCR), possibly due to complexity of the target region in view of PCR amplification. In order to further confirm donor integration in HDFs, 3′ junction PCR with three donor-specific forward primers (Donor-FP, -FP3, and -FP5) keeping the same reverse primer as in FIG. 2h (Outer-RP) was performed. The expected amplicon sizes were 8442 (A), 6643 (B), and 4758 bp (C), respectively (FIGS. 7a and 7c). Due to the upper amplicon size limit of DNA polymerase, only amplicons of about 4758 bp in reaction C containing the primer set “Donor-FP5 and Outer-RP” (FIG. 7a) were found.

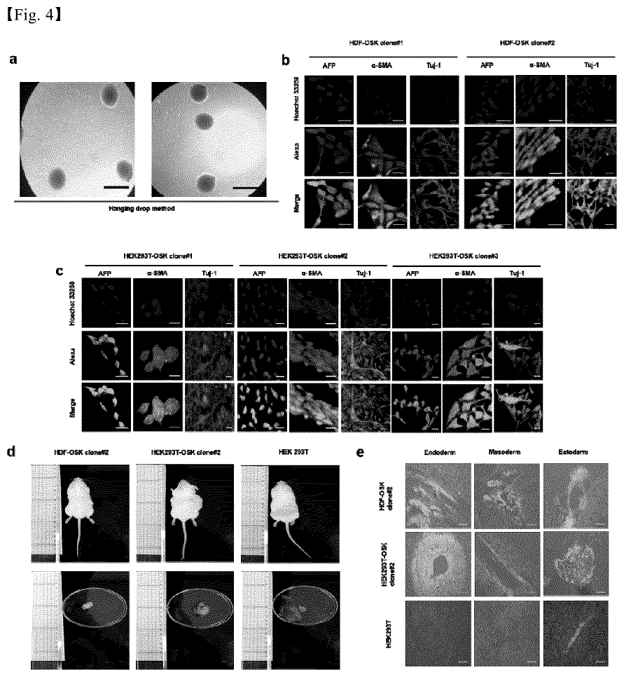

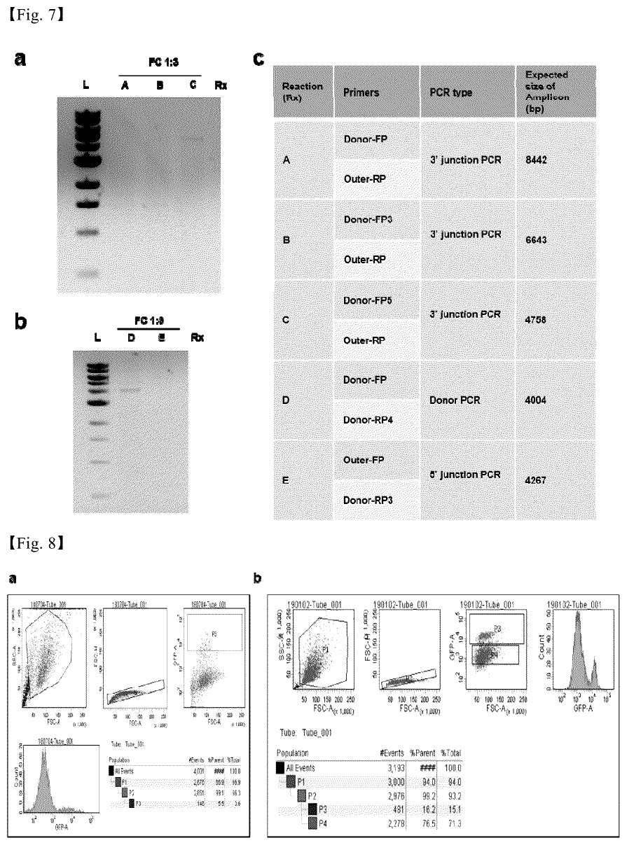

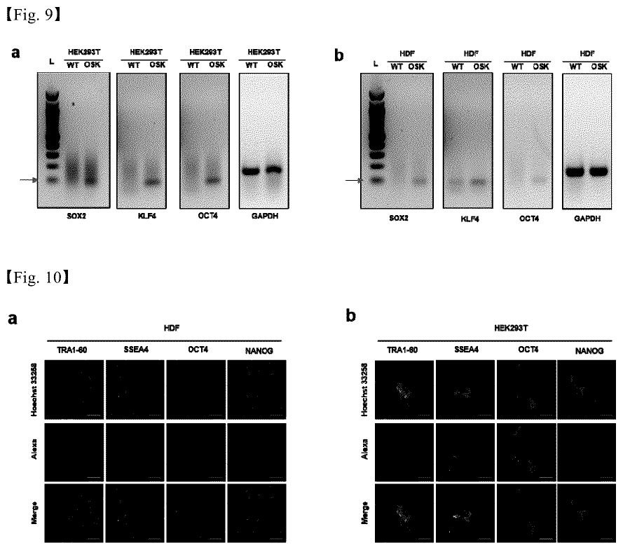

The presence of the donor cassette in HDFs was further confirmed using a donor-specific primer set (Donor-FP, and -RP4) in reaction D, but even after changing the reverse primer (Donor-RP3) in reaction E, no amplicon was observed after 5′ junction PCR. This finding indicates an obstacle to amplification from the 5′ end. Respective correct clones were selected from HEK293T cells and HDFs and designated HEK293T-OSK and HDF-OSK. Flow cytometry was performed to enrich DOX-induced GFP-positive HEK293T-OSK cells (FIG. 8a) and HDF-OSK cells (extended data FIG. 4b). In order to confirm distinct expression of transcripts for each reprogramming factor, RT-PCR analysis of OCT4, SOX2, and KLF4 transcripts in total mRNA isolated from induced HEK293T-OSK cells (FIGS. 2i and 9a) and HDF-OSK cells (FIGS. 2j and 9b) was performed. Also, GFP signals were recognized in differentiated cells only upon DOX induction, thus confirming induced expression of the donor cassette even after integration in CASH-1 (FIG. 2k).

### Example 4-2: Reprogramming-Vector Construction

For integrated expression of reprogramming factors, the donor cassette was designed to be specific to the CASH-1 site. Briefly, open reading frames (ORFs) of OCT4, SOX2, KLF4, and EGFP were used to construct a single polycistronic expression cassette for separate expression of each of these genes. This was performed by removing stop codons from the end of each gene (except for EGFP) and separating the same through insertion of 2A peptide sequences for ribosome skipping. EGFP was included to serve as an expression marker. For controlled expression of the reprogramming factors, the promoter sequence of the Tet-on system was used. A blasticidin (BSD) resistance gene was inserted as a eukaryotic selection marker. For precise integration in CASH-1, the donor cassette was flanked by 800 bp left-and right-hand sequences homologous to CASH-1. The aforementioned fragments were also separated by various restriction sites, separators, and tails. The whole designed cassette was inserted into pUC57-Amp serving as a cloning vector. The customized gene synthesis and cloning service of Synbio Technologies were used to construct the donor vector.

### Example 4-3: Donor Cassette Knock-In Optimization

Unsynchronized and cell cycle-synchronized HEK293T cells were transfected with the gRNA/Cas9 vector and the donor vector at a molar ratio of 5:1, 1:1, or 1:3. Both circular and linear forms of the donor vector were tested with the wild-type and nickase versions of Cas9, individually. In order to linearize the donor vector, it was double-digested with BamHI-HF (NEB #R3136) and EcoRV-HF (NEB #R3195) and gel-purified using a PureLink™ Quick Gel Extraction Kit (Invitrogen) according to the manufacturer's protocol. For transfection, the cells were allowed to grow to 70% confluency in a 12-well plate, after which a 30-minute-preincubated mixture of 3 μl of Lipofectamine 2000 (Invitrogen) and 1.5 μg of total DNA in Opti-MEM (Gibco) was added dropwise to the cells. After treatment for 6 hours, the medium was replaced with fresh complete DMEM and the transfected cells were allowed to grow for an additional 72 hours. Transfection-positive cells were selected in medium supplemented with 10 μg/ml blasticidin (InvivoGen) for 2 weeks.

Unsynchronized and cell cycle-synchronized HDFs were transfected with the gRNA/Cas9 vector and the donor vector at a molar ratio of 1:1 or 1:3. Both circular and linear forms of the donor vector were tested with the nickase version of Cas9, separately. Procedures for linearization, transfection, and selection were similar to those for HEK293T cells. The genetically modified HEK293T and HDF cell lines were named as HEK 293T-OSK and HDF-OSK, respectively.

### Example 5: Junction PCR

Genomic DNA was isolated from the selected cells using a DNA Tissue Kit (Kurabo, Japan) according to the manufacturer's protocol. The 5′ and 3′ junction PCRs were performed with the GoTaq Green Master Mix (Promega #M7122) and relevant primers (Table 3). The thermocycler program was set as follows: 1 cycle at 95° C. for 5 minutes; 30 cycles at 95° C. for 30 seconds, 58° C. for 30 seconds, and 72° C. for 3 minutes; 1 cycle at 72° C. for 10 minutes; and holding at 4° C. The amplicons were analyzed through electrophoresis on a 1% ethidium bromide (Invitrogen)-containing agarose gel and visualized under UV light.

### Example 6: Flow Cytometry

Stable cell lines were allowed to grow to 95% confluency in culture medium supplemented with 2 μg/ml DOX (Sigma) and 10 μg/ml blasticidin (InvivoGen). On the day of sorting, the cells were trypsinized with 0.05% trypsin-EDTA solution (Gibco) and centrifuged at 415 rcf for 3 minutes, and the concentration thereof was adjusted to 10⁷ cells/ml after cell counting. The cells were then sorted based on the GFP signal intensity through flow cytometry (FACSAria III, BD Biosciences). Data processing was performed using FACSDiva software (BD Biosciences). The sorted cells were then collected into a new tube containing medium, centrifuged at 415 rcf for 3 minutes, and seeded in a 6 cm dish containing growth medium.

### Example 7-1: Production and Characterization of iPSCs

HDF-OSK and HEK293T-OSK cells were used for iPSC production (FIG. 3a). These cells were treated with DOX-containing reprogramming medium for 12 days, after which morphological changes were observed in both types of cells. The clones increased in size after 24 days, and were manually harvested and transferred to a Matrigel-coated dish containing stem cell maintenance medium. The transferred clones retained the morphology thereof and became larger (FIG. 3b). Six morphologically changed clones derived from HDF-OSK cells were collected and three from HEK293T-OSK cells were collected. These clones were analyzed for mRNA expression of endogenous pluripotent markers including OCT4, SOX2, KLF4, c-MYC, and NANOG. All iPSC clones showed similar expression of these markers to the positive control: human embryonic stem cells (FIG. 3c) as previously reported (Kim, J. B. et al. Nature 461, 649-653 (2009); Kim, J. B. et al. Cell 136, 411-419 (2009)). Two iPSC clones from HDF-OSK cells and three from HEK293T-OPK cells were collected for further analysis of pluripotent markers through immunofluorescence staining. Signals for pluripotent markers, namely NANOG, OCT4, SSEA4, and TRA1-60, were detected in clones derived from HDF-OSK cells (FIG. 3d) and clones from HEK293T-OPK cells (FIG. 3e). In contrast, no signals for any markers could be detected in HDF (extended FIG. 6a) and HEK293T (extended data FIG. 6b) as negative controls. Production of fully reprogrammed iPSCs is associated with silencing of the transgene, and thus, silencing of the transgene was observed in morphologically changed iPSCs (FIG. 3f).

### Example 7-2: Production and Maintenance of iPSCs

HDF-OSK and HEK293T-OSK cells were seeded in a gelatin-coated (0.2%) 6-well plate at a density of 9x10 4 /well and allowed to grow in normal medium (as described above) for 24 hours. After 24 hours, the medium was replaced with reprogramming medium: knockout DMEM/F12 (Gibco) supplemented with 20% knockout serum replacement (KSR; Gibco), 2 mM L-glutamine (Gibco), 0.1 mM nonessential amino acids (Gibco), 0.1 mM β-mercaptoethanol (Gibco), 50 U/ml penicillin, 50 μg/ml streptomycin (Hyclone), 10 ng/ml basic fibroblast growth factor (bFGF; Peprotech), 2 μg/ml DOX, and 10 μg/ml blasticidin. The medium was refreshed daily until emergence of morphological changes of the cells. The morphologically changed colonies were manually harvested and transferred to a Matrigel-coated 6-well plate containing TESR-E8 maintenance medium (Stemcell) and 10 μM ROCK inhibitor (Stemcell). The next day, the medium was replaced with fresh maintenance medium without the ROCK inhibitor, and the latter medium was refreshed every 2 days until the colonies grew enough to be subcultured. For subculturing, a mechanical approach with a cell scraper was used.

### Example 8-1: Differentiation Potential of iPSCs

For spontaneous in-vitro differentiation of iPSCs, a hanging drop method was used to form embryoid bodies (FIG. 4a). These embryoid bodies were then differentiated in differentiation medium, which was confirmed through immunofluorescence staining for markers of respective germ layers, namely AFP (for endoderm), α-SMA (for mesoderm), and TUJ-1 (for ectoderm). All clones derived from HDF-OSK cells (FIG. 4b) and HEK-OSK cells (FIG. 4c) tested positive for all three germ layers. In order to evaluate pluripotency in vivo, HDF-OSK clone #2 and HEK293T-OSK clone #2 were injected into the dorsal flank of SCID (severe combined immunodeficiency) mice. Also, due to the cancerous nature of HEK293T cells, HEK 293T cells were injected as a negative control. Seven weeks after injection, tumor formation was observed at the injected site for each type of clone (FIG. 4d). The negative control and all clones developed tumors, but teratomas formed by the control cells were only positive for ectoderm, unlike teratomas formed by the iPSC clones. These tumors tested positive for markers of all three germ layers in immunohistochemical analysis (FIG. 4e).

### Example 8-2: In-Vitro Differentiation of iPSCs

A hanging drop method was used for formation of embryoid bodies. The harvested iPSCs were counted, the concentration thereof was adjusted to 1000 cells/20 μl of bFGF-free culture medium, and the cells were incubated in a hanging drop on the lid of a Petri dish for 2 days. The resulting embryoid bodies were suspension-cultured for 5 days in DMEM supplemented with 10% FBS, 1 mM L-glutamine, and 1% nonessential amino acid solution. The embryoid bodies were then transferred to 12-well plates and allowed to grow on gelatin-coated coverslips for another 7 days, with refreshment of the medium every other day. Finally, the coverslips were removed, followed by processing for immunocytochemical analysis of the germ layers.

### Example 9: Immunocytochemistry and Immunohistochemistry

For immunocytochemical analysis, the iPSCs or embryoid bodies that spontaneously differentiated were allowed to grow in suitable media on glass coverslips placed in a 12-well plate. The grown cells were washed with 1×PBS, fixed, permeabilized in cold methanol (Samchun Chemicals, Korea) for 10 minutes, washed with PBS, and blocked with a 3% BSA solution in PBS (Thermo Fisher Scientific, Inc.) for 30 minutes. Thereafter, the cells were incubated overnight at 4° C. with primary antibodies. The next day, the cells were rigorously washed and incubated with Alexa Fluor 488- or Alexa Fluer 546-conjugated secondary antibodies (Invitrogen, Carlsbad, CA, USA) at room temperature for 1 hour. After washing with PBS, nuclei were stained with a Hoechst 33258 solution (5 μM; Sigma-Aldrich) for 10 minutes. For staining of pluripotent markers of iPSCs, the following primary antibodies were used: anti-TRA1-60 (1:500; Abcam), anti-SSEA4 (1:500; Abcam), anti-OCT4A (1:1000; Cell Signaling Technology), and anti-NANOG (1:1000; Cell Signaling Technology). In order to detect germ layer markers of differentiation, the following primary antibodies were used: anti-AFP (for endoderm; 1:100; Santa Cruz Biotechnology), anti-SMA (for mesoderm; 1:250, Sigma), and anti-TUJ-1 (for ectoderm; 1:250; Abcam).

For immunohistochemical staining, formalin-fixed paraffin-embedded tissue samples were split into monolayers of cells, mounted on glass slides, and subjected to deparaffinization, rehydration, and antigen retrieval according to a typical protocol. Tissue samples were fixed, permeabilized, probed with antibodies specific to germ layer markers, and finally stained for nuclear DNA according to the protocol mentioned above. The antibodies applied to identify the derivatives of individual germ layers are mentioned above. All images were captured using a fluorescence microscope (Olympus IX53; Olympus Corporation, Tokyo, Japan).

### Example 10: RT-PCR and Quantitative RT-PCR

For both types of PCR, total RNA was isolated using a TRI Reagent® Solution (Sigma-Aldrich) according to the manufacturer's protocol. Impurities made up of genomic DNA were removed from RNA samples using a TURBO DNA-free™ Kit (Thermo Fisher Scientific Inc.). The purified RNA was reverse-transcribed using an iScript cDNA Synthesis Kit (BioRad). For RT-PCR, target genes were amplified using GoTaq® Green Master Mix (Promega) under the following thermal cycling conditions: 95° C. for 5 minutes followed by 25 cycles of 95° C. for 10 seconds, 58° C. for 10 seconds, and 72° C. for 20 seconds. For quantitative RT-PCR, expression of target genes was quantified using Light Cycler 480 SYBR Green I Master Mix (Roche) under the following thermal cycling conditions: 95° C. for 5 minutes followed by 45 cycles of 95° C. for 10 seconds, 58° C. for 10 seconds, and 72° C. for 20 seconds. The amount of each mRNA was normalized to that of GAPDH mRNA, and the relevant mRNA of untreated cells served as a negative control. Error bars shown are averages with standard deviations of three independent experiments. The primers used are listed in Table 3.

### Example 11: Western Blotting

The cells were collected by centrifugation and total protein was extracted with Whole-Protein Extraction Solution (M-PER; Thermo Fisher Scientific Inc.) supplemented with a protease and phosphatase inhibitor cocktail (Thermo Fisher Scientific Inc.). The mixture was centrifuged at 16,000×g for 10 minutes, and protein in the supernatant was quantified using a BCA Kit (Sigma-Aldrich Co. LLC). Protein samples (20 μg) were loaded onto an SDS-polyacrylamide gel, transferred to a Hybond-ECL nitrocellulose membrane (Amersham Pharmacia Biotech, Inc., Piscataway, NJ, USA), and blocked with 5% nonfat dry milk, followed by immunoblotting overnight at 4° C. with primary antibodies against OCT4A (2890, Cell Signaling Technology), SOX2 (ab97959, Abcam), and KLF4 (ABS1514, Merck). The next day, the membrane was washed with PBST, incubated with horseradish peroxidase-conjugated anti-mouse or anti-rabbit secondary antibodies (Thermo Fisher Scientific Inc.), treated with a SuperSignal West Pico ECL solution (Thermo Fisher Scientific Inc.), and visualized using a Fuji LAS-3000 system (Fujifilm, Tokyo, Japan).

### Example 12: Teratoma Formation Assay

Human iPSCs (¼ of cells from a 100 mm confluent dish) were injected subcutaneously into two parts of the dorsal flank of SCID mice (male, 8 weeks old). Seven weeks after injection, teratomas were fixed in 4% paraformaldehyde for 48 hours and embedded in paraffin. Tissue sections were prepared and processed for immunohistochemical analysis as described above.

### INDUSTRIAL APPLICABILITY

According to the present invention, a method of producing induced pluripotent stem cells is capable of preventing off-target mutagenesis, safely and specifically integrating a reprogramming factor into the target site, and ensuring consistent expression by controlling the reprogramming factor with doxycycline, thus efficiently producing induced pluripotent stem cells. In addition, induced pluripotent stem cells produced through the above method can show expression of pluripotent markers similar to those of human embryonic stem cells, and can differentiate into derivatives of all three germ layers.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents thereto.

### Sequence List Free Text

An electronic file is attached.

1. A method of producing induced pluripotent stem cells comprising inserting a nucleic acid encoding a reprogramming factor into CASH-1 (CRISPR/Cas9-accessible safe harbor-1) of somatic cells using a CRISPR/Cas system, wherein the CRISPR/Cas system contains a delivery vehicle comprising nucleic acids encoding a gRNA (guide RNA) and a Cas protein, and the gRNA comprises gRNA 1 represented by SEQ ID NO: 1 and gRNA 2 represented by SEQ ID NO: 2.

2. The method according to claim 1, wherein the CASH-1 corresponds to position 188,083,272 of chromosome 1 of somatic cells.

3. The method according to claim 1, wherein the delivery vehicle is at least one selected from the group consisting of a vector, a ribonucleoprotein (RNP) complex, a carrier, and an exosome.

4. The method according to claim 3, wherein the nucleic acids encoding the gRNA and the Cas protein are contained in identical or different delivery vehicles.

5. (canceled)

6. The method according to claim 3, wherein the vector is a viral vector selected from the group consisting of an adeno-associated viral vector (AAV), an adenoviral vector (AdV), a lentiviral vector (LV), and a retroviral vector (RV); or an episomal vector comprising a viral replicon.

7. The method according to claim 6, wherein the episomal vector comprises an origin of replication (Ori) of Simian virus 40 (SV40), bovine papilloma virus (BPV), or Epstein-Barr nuclear antigen (EBV).

8. The method according to claim 3, wherein the vector is pX330-U6-Chimeric_BB-CBh-hSpCas9 or pX335-U6-Chimeric_BB-CBh-hSpCas9n(D10A).

9. The method according to claim 1, wherein the Cas protein is a Cas3, Cas9, Cpf1, Cas6, C2c12, or C2c2 protein.

10. The method according to claim 1, wherein the Cas protein is derived from a genus of microorganisms containing an ortholog of a Cas protein selected from the group consisting of Corynebacter, Sutterella, Legionella, Treponema, Filifactor, Eubacterium, Streptococcus, Lactobacillus, Mycoplasma, Bacteroides, Flaviivola, Flavobacterium, Azospirillum, Gluconacetobacter, Neisseria, Roseburia, Parvibaculum, Staphylococcus, Nitratifractor, Corynebacterium, and Campylobacter, and is isolated therefrom or recombinant.

11. (canceled)

12. (canceled)

13. The method according to claim 1, wherein the nucleic acid encoding the reprogramming factor is inserted using a vector comprising the nucleic acid encoding the reprogramming factor.

14. The method according to claim 13, wherein the vector comprising the nucleic acid encoding the reprogramming factor is a viral vector or a non-viral vector.

15. The method according to claim 14, wherein the viral vector is selected from the group consisting of an adeno-associated viral vector (AAV), an adenoviral vector (AdV), a lentiviral vector (LV), and a retroviral vector (RV), or wherein the non-viral vector is a plasmid vector or an episomal vector.

16. (canceled)

17. The method according to claim 15, wherein the plasmid vector is pUC57-Amp.

18. The method according to claim 1, wherein the reprogramming factor is at least one selected from the group consisting of OCT4, SOX2, KLF4, c-MYC, NANOG, SSEA4, and TRA1-60.

19. The method according to claim 18, wherein the reprogramming factor is at least one selected from the group consisting of OCT4, SOX2, and KLF4.

20. The method according to claim 1, wherein expression of the reprogramming factor is controlled with doxycycline.

21. The method according to claim 13, wherein a molar ratio of the vector comprising the nucleic acids encoding the gRNA and the Cas protein to the vector comprising the nucleic acid encoding the reprogramming factor is 1:1-1:3.

22. The method according to claim 21, wherein the vector comprising the nucleic acid encoding the reprogramming factor is linear.

23. The method according to claim 1, wherein the somatic cells are mouse fibroblasts, human fibroblasts (HDFs), human kidney cells (HEK293T), human keratinocytes, or human peripheral blood cells.

24. The method according to claim 23, wherein the somatic cells are human fibroblasts (HDFs) or human kidney cells (HEK293T).
